(19) United States
(12) Patent Application Publication
(10) Pub. No.: US 2026/0148379 A1
(43) Pub. Date: May 28, 2026

(54) X-RAY CT APPARATUS, MEDICAL IMAGE PROCESSING APPARATUS, AND MEDICAL IMAGE PROCESSING METHOD

(71) Applicant: CANON MEDICAL SYSTEMS CORPORATION, Otawara-shi (JP)

(72) Inventors: Gakuto AOYAMA, Kawasaki (JP); Yoshinori HIRANO, Nasushiobara (JP)

(73) Assignee: CANON MEDICAL SYSTEMS CORPORATION, Otawara-shi (JP)

(21) Appl. No.: 19/383,881

(22) Filed: Nov. 10, 2025

(30) Foreign Application Priority Data
Nov. 22, 2024 (JP) 2024-203653

(51) Int. Cl. G06T 7/00; A61B 6/00; A61B 6/03; A61B 6/46

(52) U.S. Cl. CPC G06T 7/0012; A61B 6/032; A61B 6/463; A61B 6/5205; G06T 2207/10116

(57) ABSTRACT

An X-ray CT apparatus or medical image processing apparatus according to embodiments, which includes an X-ray tube that generates X-rays and an X-ray detector that detects the X-rays emitted from the X-ray tube and passing through a subject, and generates medical image data based on detection data detected by the X-ray detector, includes processing circuitry. The processing circuitry inputs the medical image data to a generative model, acquires generated image data generated by the generative model, measures a measurement range using the generated image data, and outputs predetermined information in a case where at least a part of the measurement range is included in a generated region in the generated image data.

### CROSS-REFERENCE TO RELATED APPLICATIONS

This application is based upon and claims the benefit of priority from Japanese Patent Application No. 2024-203653, filed on November 22, 2024; the entire contents of all of which are incorporated herein by reference.

### FIELD

Embodiments described herein relate generally to an X-ray CT apparatus, a medical image processing apparatus, and a medical image processing method.

### BACKGROUND

In recent years, medical image diagnostic apparatuses such as X-ray computed tomography (CT) apparatuses scan a subject to generate medical image data. The medical image data may contain artifacts due to metal or beam hardening. In a case where the medical image data contains noise such as artifacts in this way, a generative model can generate image data with reduced artifacts.

However, an image generated by the generative model is not identical to the original image obtained by scanning the subject. Therefore, it is desirable for a medical professional to read images while grasping that the images are generated by a generative model.

### BRIEF DESCRIPTION OF THE DRAWINGS

FIG. 1 is a diagram illustrating an exemplary medical image processing system according to a first embodiment;

FIG. 2 is a diagram illustrating an exemplary processing flow by a model control function;

FIG. 3 is a diagram illustrating an exemplary generated image in which a generated region can be identified;

FIG. 4 is a diagram illustrating an exemplary measurement operation process executed by a medical image processing apparatus according to the first embodiment;

FIG. 5 is a diagram illustrating an exemplary generated image in which a generated region can be identified;

FIG. 6 is a diagram illustrating an exemplary medical image processing system according to a second embodiment;

FIG. 7 is a diagram illustrating an exemplary generated image in which a generated region can be identified;

FIG. 8 is a diagram illustrating an exemplary medical image processing system according to a third embodiment;

FIG. 9 is a diagram illustrating exemplary measurement using the generated image;

FIG. 10 is a diagram illustrating exemplary measurement using the generated image;

FIG. 11 is a diagram illustrating exemplary measurement using the generated image;

FIG. 12 is a diagram illustrating exemplary measurement using the generated image;

FIG. 13 is a diagram illustrating exemplary measurement using the generated image;

FIG. 14 is a diagram illustrating exemplary measurement using the generated image;

FIG. 15 is a diagram illustrating exemplary measurement using three-dimensional medical image data; and

FIG. 16 is a diagram illustrating an exemplary configuration of a medical information processing apparatus according to an eighth modification.

### DETAILED DESCRIPTION

An X-ray CT apparatus or medical image processing apparatus according to embodiments, which includes an X-ray tube that generates X-rays and an X-ray detector that detects the X-rays emitted from the X-ray tube and passing through a subject, and generates medical image data based on detection data detected by the X-ray detector, includes processing circuitry. The processing circuitry inputs the medical image data to a generative model, acquires generated image data generated by the generative model, measures a measurement range using the generated image data, and outputs predetermined information in a case where at least a part of the measurement range is included in a generated region in the generated image data.

The X-ray CT apparatus, the medical image processing apparatus, and the medical image processing method according to the present embodiments will be described below with reference to the drawings. In the following embodiments, parts denoted by the same reference signs are assumed to operate in the same way, and duplicate explanations are omitted as appropriate.

### First Embodiment

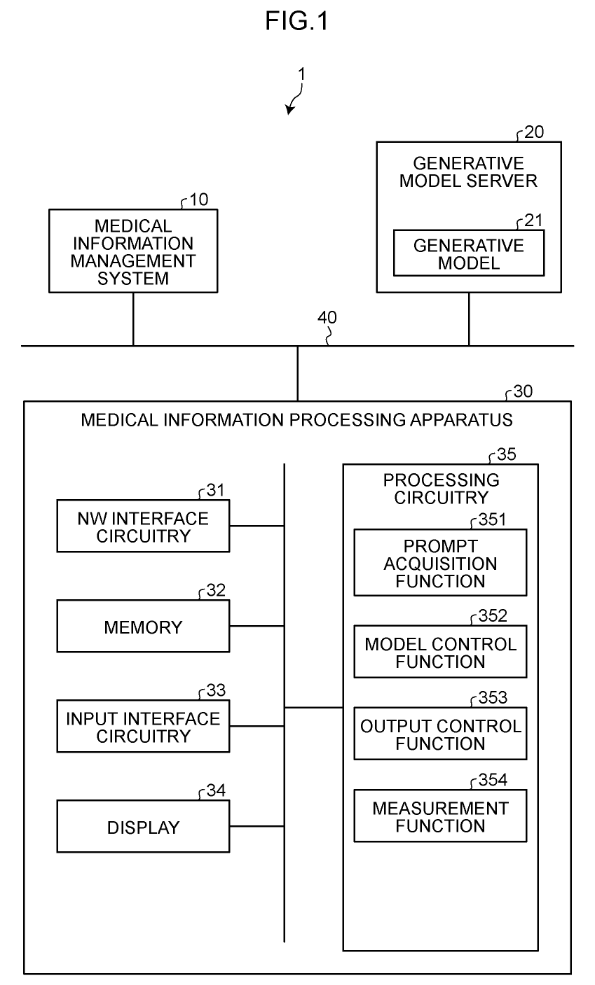

FIG. 1 is a diagram illustrating an example of a medical information processing system 1 according to a first embodiment. As illustrated in FIG. 1, the medical information processing system 1 has a medical information management system 10, a generative model server 20, and a medical information processing apparatus 30. The medical information processing system 1 is a system having a generative model server 20 that generates at least part of medical image data, and a medical information processing apparatus 30 that displays an image generated by a generative model 21 of the generative model server 20. The medical information management system 10, the generative model server 20, and the medical information processing apparatus 30 are communicatively connected via a network 40 such as a local area network (LAN). The medical information processing system 1 illustrated in FIG. 1 is an example and may have other apparatuses and systems.

The medical information management system 10 is a system that saves and manages medical information. For example, the medical information management system 10 is implemented by one or more computer devices such as servers and personal computers. The medical information is, for example, medical image data or information that includes medical image data.

The medical information management system 10 may be, for example, an electronic medical record system, a hospital information system (HIS), a laboratory information system (LIS), a radiology information system (RIS), a picture archiving and communication system (PACS), or the like. The medical information management system 10 may include more than one of the systems, apparatuses, and the like listed above.

The generative model server 20 includes the generative model 21. The generative model server 20 also has a memory and a processor. The generative model 21 is, for example, stored in the memory of the generative model server 20 and operated by the processor. The configuration of the generative model server 20 is not particularly limited, and any known configuration can be employed.

When accepting a prompt, the generative model 21 generates generated image data corresponding to the prompt. The generative model 21 is, for example, a trained model such as a multimodal model (MMM) or a large language model (LLM) that can handle multiple types of data such as image data. The generative model 21 is an integrated, trained model that can process multiple types of modalities (data types) such as text, images, audio, and numerical values at once. For example, the generative model 21 may search information such as a database owned by the user using the generative model 21 to generate generated image data, like retrieval-augmented generation (RAG).

Based on the prompt, the generative model 21 generates the generated image data in which at least part of the input medical image data is generated. For example, based on the prompt, the generative model 21 generates an image with reduced noise such as artifacts contained in the medical image data. The generative model 21 may generate not only an image with reduced noise such as artifacts but also information outside an imaging region in the medical image data, or may generate an image in which a device related to treatment in the medical image data is removed. The generative model 21 generates information of a region outside the imaging region (imaging range or reconstruction range) as the generation of information outside the imaging region. For example, the generative model 21 generates an image corresponding to the head region for an image captured by setting an imaging range from the neck to the waist. For example, the generative model 21 generates a region outside an image (for example, a region of parts of ribs or lungs if they are cropped off) for an enlarged reconstructed image of the heart. The device related to treatment is a device such as a coil or stent to be inserted into a blood vessel or a clip to pinch a blood vessel. For example, the generative model 21 generates an image in which the device related to treatment is not visualized from an image in which the device related to treatment is visualized, as the generation of an image in which the device related to treatment is removed. For example, the generative model 21 generates an image by inferring information free from the device related to treatment from information about the surroundings of the device related to treatment.

The generative model 21 may be held not only by the generative model server 20 but also by other apparatuses. For example, the generative model 21 may be held by the medical information processing apparatus 30.

The medical information processing apparatus 30 is an apparatus operated by a medical professional. For example, the medical information processing apparatus 30 accepts an operation to specify a measurement target of a subject included in a medical image. The medical information processing apparatus 30 is an example of the medical image processing apparatus. For example, the medical information processing apparatus 30 is implemented by a computer device such as a personal computer.

The medical information processing apparatus 30 includes network (NW) interface circuitry 31, a memory 32, input interface circuitry 33, a display 34, and processing circuitry 35.

The NW interface circuitry 31 is connected to the processing circuitry 35 and controls transmission and communication of various data to and from each apparatus connected via the network 40. For example, the NW interface circuitry 31 is implemented by a network card, a network adapter, a network interface controller (NIC), or the like.

The memory 32 is connected to the processing circuitry 35 and stores therein various information in advance. The memory 32 stores therein various computer programs. The memory 32 is, for example, a non-volatile storage device, such as a hard disk drive (HDD), a solid state drive (SSD), or an integrated circuit storage device, that stores therein various information. The memory 32 may be a drive device, other than an HDD or an SSD, that reads and writes various information from/into a portable storage medium such as a compact disc (CD), a digital versatile disc (DVD), or a flash memory, or a semiconductor memory device such as a random access memory (RAM). The memory 32 is an example of a memory unit.

The input interface circuitry 33 is connected to the processing circuitry 35 to convert an input operation accepted from the operator (medical professional) into an electrical signal and output the electrical signal to the processing circuitry 35. Specifically, the input interface circuitry 33 converts the input operation accepted from the operator into an electrical signal and outputs the electrical signal to the processing circuitry 35. For example, the input interface circuitry 33 is implemented by a trackball, a switch button, a mouse, a keyboard, a touchpad that allows for an input operation by touching an operation surface, a touchscreen that integrates a display screen and a touchpad, a non-contact input circuit using an optical sensor, an audio input circuit, and the like. As used herein, the input interface circuitry 33 is not limited to those with physical operating components, such as a mouse and a keyboard. For example, an electrical signal processing circuit that receives an electrical signal corresponding to an input operation from an external input device installed separately from the apparatus and outputs the electrical signal to control circuitry is also an example of the input interface circuitry 33.

The display 34 is connected to the processing circuitry 35 and displays various information and various image data output from the processing circuitry 35. For example, the display 34 is implemented by a liquid crystal display, a cathode ray tube (CRT) display, an organic EL display, a plasma display, a touch panel, or the like.

The processing circuitry 35 controls the operation of the entire medical information processing apparatus 30. The processing circuitry 35 has, for example, a prompt acquisition function 351, a model control function 352, an output control function 353, and a measurement function 354. In the embodiment, each of the processing functions performed by the prompt acquisition function 351, the model control function 352, the output control function 353, and the measurement function 354, which are components, is stored in the memory 32 in the form of a computer program executable by a computer. The processing circuitry 35 is a processor that reads computer programs from the memory 32 and executes the computer programs to implement a function corresponding to each of the computer programs. In other words, the processing circuitry 35 in a state in which the computer programs are read out has each function depicted in the processing circuitry 35 in FIG. 1.

In FIG. 1, a single processor implements the prompt acquisition function 351, the model control function 352, the output control function 353, and the measurement function 354. However, a plurality of independent processors may be combined to construct the processing circuitry 35 so that each processor executes a computer program to implement a function. In FIG. 1, a single memory such as the memory 32 stores therein the computer program corresponding to each processing function. However, a plurality of memories may be distributed and allocated, and the processing circuitry 35 may be configured to read a corresponding computer program from each individual memory.

The term "processor" used in the foregoing description means circuitry such as a central processing unit (CPU), a graphical processing unit (GPU), an application specific integrated circuit (ASIC), or a programmable logic device (e.g., simple programmable logic device (SPLD), complex programmable logic device (CPLD), and a field programmable gate array (FPGA)). The processor reads and executes a computer program saved in the memory 32 to implement a function. Instead of saving a computer program in the memory 32, the computer program may be embedded directly in the circuitry of the processor. In this case, the processor reads and executes a computer program embedded in the circuitry to implement a function.

The prompt acquisition function 351 acquires a prompt to be input to the generative model 21. In more detail, the prompt acquisition function 351 controls the input interface circuitry 33 to acquire a prompt indicating instruction contents for the generative model 21.

The prompt is, for example, data indicating instruction contents for the generative model 21. For example, the prompt is information such as text data and image data. The prompt includes instruction content information and instruction target information. The instruction content information is information indicating instruction contents for the generative model 21. For example, the instruction content information is text data that gives an instruction to reduce artifacts contained in medical image data. The instruction content information may include artifact location information indicating the location in the medical image data where artifacts to be reduced are present. The artifact location information may identify the location of artifacts by the subject's part, such as the lung field region, or may identify the location where artifacts are present by coordinates, or may identify the location of artifacts by other methods.

The instruction target information is information indicating input image data serving as an instruction target. For example, the input image data is medical image data having a region, such as artifacts, to be processed by the generative model 21. For example, the input image data is CT image data. However, the input image data is not limited to CT image data, but may be X-ray image data, magnetic resonance image data, ultrasound image data, or other medical image data.

For example, the instruction target information may be identification information to identify the input image data serving as an instruction target, or a path indicating the location where the input image data is saved. The instruction target information may be the input image data itself. In this case, the prompt acquisition function 351 acquires a prompt including medical image data.

The model control function 352 controls the generative model 21. In more detail, the model control function 352 inputs the prompt acquired by the prompt acquisition function 351 into the generative model 21. The model control function 352 acquires the generated image data output from the generative model 21. The model control function 352 is an example of an image acquisition unit. For example, the model control function 352 acquires the generated image data in which artifacts contained in the input image data are reduced by the generative model 21 that generates an image with reduced artifacts.

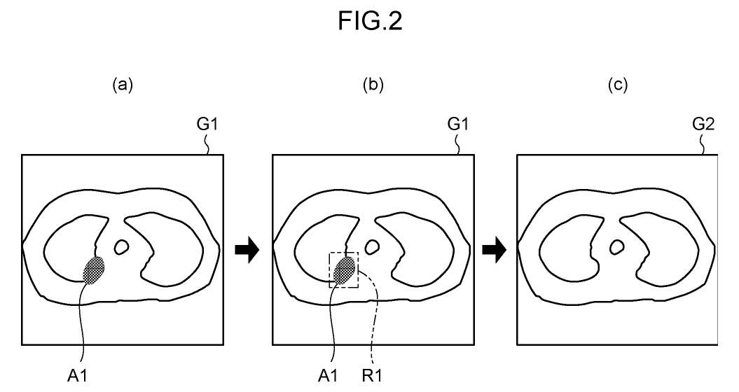

FIG. 2 is a diagram illustrating an exemplary processing flow by the model control function 352. FIG. 2(a) is a diagram illustrating an example of an input image G1 based on input image data. FIG. 2(b) is a diagram illustrating an example of an image generation region R1 to be processed by the generative model 21 on the input image G1. FIG. 2(c) is a diagram illustrating an example of a generated image G2 based on the generated image data output by the generative model 21 receiving the input image data.

As illustrated in FIG. 2(a), the input image G1 has an artifact region A1, which is a region of artifacts. As illustrated in FIG. 2(b), the generative model 21 generates image data for the image generation region R1 including the artifact region A1. In other words, the image generation region R1 is a region where an image is generated by the generative model 21. For example, the image generation region R1 is a region including artifacts, a region outside the imaging region in the input image data, or a region including a device related to treatment in the input image data. The generative model 21 then generates an image in which artifacts are reduced, an image in which information outside the imaging region is generated, or an image in which a device related to treatment is removed. FIG. 2(b) illustrates an example in which a rectangular region (ROI: Region of Interest) is set as the image generation region R1, but the shape of the image generation region R1 is not limited to this. For example, the shape of the image generation region R1 may be circular, a polygon other than a rectangle, or a shape that follows the contour of the artifact region A1. Furthermore, the image generation region R1 may be a two-dimensional or three-dimensional region. As illustrated in FIG. 2(c), the generative model 21 outputs a generated image G2 in which an image in the image generation region R1 is generated. The model control function 352 acquires the generated image data generated by the generative model 21.

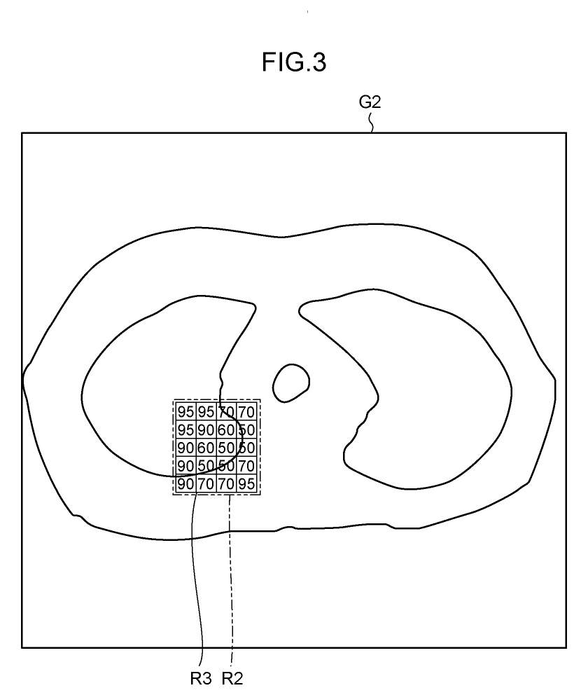

The model control function 352 may acquire image generation likelihood information if the image generation likelihood information is output from the generative model 21. The image generation likelihood information is information that has information indicating the location of a generated region R2 (see FIG. 3) corresponding to the image generation region R1 and likelihood information for each subregion which is a subdivision of the generated region R2. Here, the image generation region R1 is a region where an image is to be generated by the generative model 21 in the input image data. On the other hand, the generated region R2 is a region of an image generated by the generative model 21 in the generated image data. For example, the information indicating the location of the generated region R2 is the coordinates indicating the location of the generated region R2 in the generated image data. The likelihood information is the degree indicating the likelihood of the image of each subregion in the generated region R2 generated by the generative model 21. In other words, the image generation likelihood information is information that associates the location where each subregion of the generated region R2 is set with the likelihood of each subregion. The subregion may be of any size or shape. The subregion is set by a predetermined method and associated with the likelihood. For example, the generative model 21 sets subregions by dividing by a preset number of pixels. The generative model 21 then sets the likelihood corresponding to each subregion.

The output control function 353 outputs predetermined information when at least a part of a measurement range of the measurement function 354 is included in the generated region R2 in the generated image data. For example, the output control function 353 displays or gives a notification of the predetermined information as the output of the predetermined information. For example, the output control function 353 displays the generated image G2 on the display 34 based on the generated image data. For example, the output control function 353 displays the generated image G2 in which the generated region R2, which is the region of the image generated by the generative model 21, can identified.

FIG. 3 is a diagram illustrating an example of the generated image G2 in which the generated region R2 can be identified. For example, the output control function 353 displays a likelihood notification image R3 of the same shape as the generated region R2 on the generated region R2, based on the image generation likelihood information. The likelihood notification image R3 is an image indicating the shape of the generated region R2 and the location of the generated region R2. The likelihood notification image R3 is an image that gives a notification of the likelihood of each subregion of the generated region R2 in a numerical value. In other words, the likelihood notification image R3 is an image that makes the generated region R2 identifiable. In more detail, the likelihood notification image R3 is an image having a line that demarcates each subregion of the generated region R2 and a numerical value indicating the likelihood of each of the subregions. Specifically, the output control function 353 changes each pixel of the generated image G2 saved in the memory that is identified by the image generation likelihood information to pixel values of pixels corresponding to a line or a numerical value indicating the likelihood. The output control function 353 thus generates the generated image G2 with the likelihood notification image R3 superimposed. The output control function 353 does not display the generated region R2, but displays the likelihood notification image R3 as an image indicating the generated region R2. For the likelihood notification image R3, the output control function 353 displays the likelihood (numerical value) for each subregion of the generated region R2, but may display the likelihood (numerical value) for each pixel of the generated region R2.

As illustrated in FIG. 3, the output control function 353 displays the likelihood notification image R3 indicating the likelihood of the image generated by the generative model 21 for each of a plurality of subregions included in the generated region R2. For example, the output control function 353 displays the generated image G2 with the likelihood notification image R3 superimposed on the generated region R2. In this way, the output control function 353 displays the location of the generated region R2, which is the region of the image generated by the generative model 21, in an identifiable manner. A medical professional therefore can grasp the presence of the generated region R2 by means of the likelihood notification image R3.

The measurement function 354 measures the measurement range using the generated image G2, based on an operation accepted by the input interface circuitry 33. The measurement function 354 is an example of a measurement unit. In more detail, the measurement function 354 accepts the specification of the measurement range in the generated image G2, based on an operation accepted by the input interface circuitry 33. The measurement function 354 then measures the measurement range included in the generated image data.

In more detail, the measurement function 354 measures the specified measurement range in the generated image G2. For example, the measurement function 354 measures the size of each part of the subject or the size of shadows in the generated image G2. Specifically, the measurement function 354 measures distance, area, and volume. The measurement function 354 thus measures the volume of the extracted tumor area or the cross-sectional area or cross-sectional area ratio of the brain in brain atrophy.

Here, the output control function 353 outputs predetermined information when at least a part of the measurement range of the measurement function 354 is included in the generated region R2 in the generated image data. The output control function 353 is an example of a control unit. For example, the output control function 353 warns that the generated region R2 is being measured when the measurement range to be measured by the measurement function 354 is included in the generated region R2 in which artifacts are reduced by the generative model 21 in the generated image data generated by the generative model 21. A medical professional thus can measure the measurement range while grasping that the image is an image generated by the generative model 21. The measurement function 354 may measure the measurement range using the generated image G2 based on not only the operation accepted by the input interface circuitry 33 but also other specifications.

The output control function 353 may give the measurement function 354 a notification on control to perform re-measurement when the measurement range to be measured by the measurement function 354 is included in the generated region R2 in the generated image data. For example, the output control function 353 may give a notification to re-measure the measurement range excluding the generated region R2. The measurement function 354 may then re-measure the measurement range excluding the generated region R2.

A measurement operation process performed by the medical information processing apparatus 30 will now be described. The measurement operation process is the process of measuring the specified measurement range using the generated image data generated by the generative model 21.

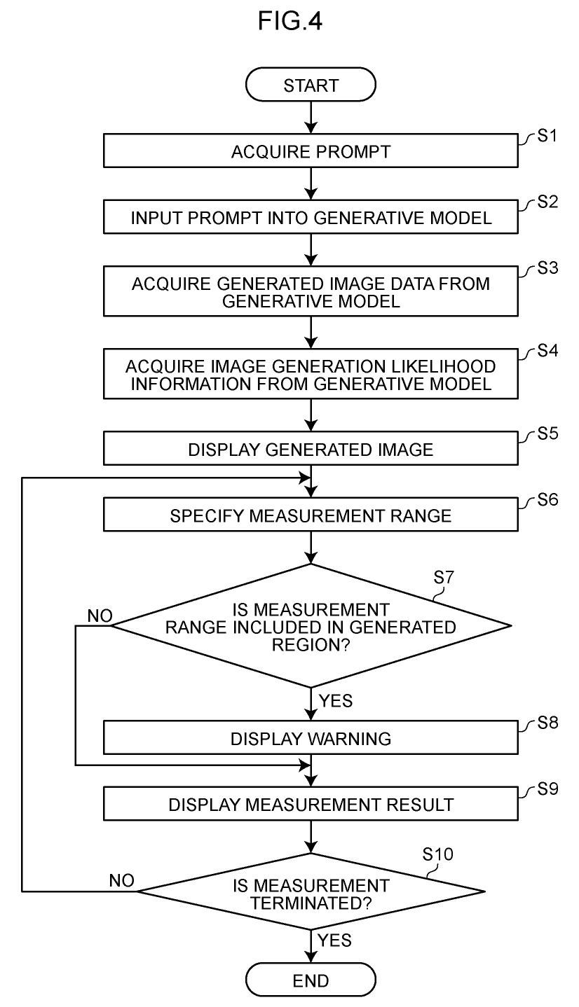

FIG. 4 is a diagram illustrating an exemplary measurement operation process executed by the medical information processing apparatus 30 according to the first embodiment.

The prompt acquisition function 351 acquires a prompt to be input to the generative model 21 (step S1).

The model control function 352 inputs the prompt acquired by the prompt acquisition function 351 into the generative model 21 (step S2).

The model control function 352 acquires, from the generative model 21, image generation data in which an image of the generated region R2 corresponding to the image generation region R1 has been generated (step S3). The model control function 352 may acquire, from the generative model 21, image generation data indicating the likelihood of each subregion which is a subdivision of the generated region R2.

The model control function 352 acquires, from the generative model 21, the image generation likelihood information indicating the likelihood of each subregion which is a subdivision of the generated region R2 (step S4). Step S4 is not essential if the generated region R2 can be grasped.

The output control function 353 displays the generated image G2 based on the generated image data generated by the generative model 21 (step S5).

The measurement function 354 accepts the specification of the measurement range based on an operation accepted by the input interface circuitry 33 (step S6).

The output control function 353 determines whether the specified measurement range is included in the generated region R2 (step S7). If the specified measurement range is included in the generated region R2 (Yes at step S7), the output control function 353 displays a warning indicating that the measurement range is included in the generated region R2 (step S8).

If the specified measurement range is not included in the generated region R2 (No at step S7), the output control function 353 does not display the warning. The output control function 353 then displays the measurement result of the measurement range measured by the measurement function 354 (step S9).

The measurement function 354 determines whether an operation to terminate the measurement is accepted (step S10). If an operation to terminate the measurement is not accepted (No at step S10), the measurement function 354 accepts the specification of the measurement range at step S6.

On the other hand, if an operation to terminate the measurement is accepted (Yes at step S10), the measurement function 354 terminates the measurement.

Consequently, the medical information processing apparatus 30 terminates the measurement operation process.

As described above, the medical information processing apparatus 30 according to the first embodiment inputs medical image data to the generative model 21 and acquires the generated image data generated by the generative model 21. The medical information processing apparatus 30 measures the measurement range using the generated image data generated by the generative model 21. The medical information processing apparatus 30 then outputs predetermined information when at least a part of the measurement range is included in the generated region R2 in the generated image data. Since the predetermined information is output, a medical professional can grasp that the measurement range is included in the image generated by the generative model 21. Thus, when a medical professional uses an image, the medical information processing apparatus 30 can allow the medical professional to easily grasp that the image or a part of the image has been generated by the generative model 21.

Furthermore, as illustrated in FIG. 3, the medical information processing apparatus 30 can also display the likelihood of each subregion by means of the likelihood notification image R3. The medical information processing apparatus 30 therefore can allow the medical professional to grasp the likelihood of the image generated by the generative model 21.

### First Modification

As illustrated in FIG. 3, the output control function 353 displays the likelihood notification image R3 indicating the likelihood of each subregion of the generated region R2 in a numerical value. However, the output control function 353 may display the likelihood not only as numerical values but also by other methods.

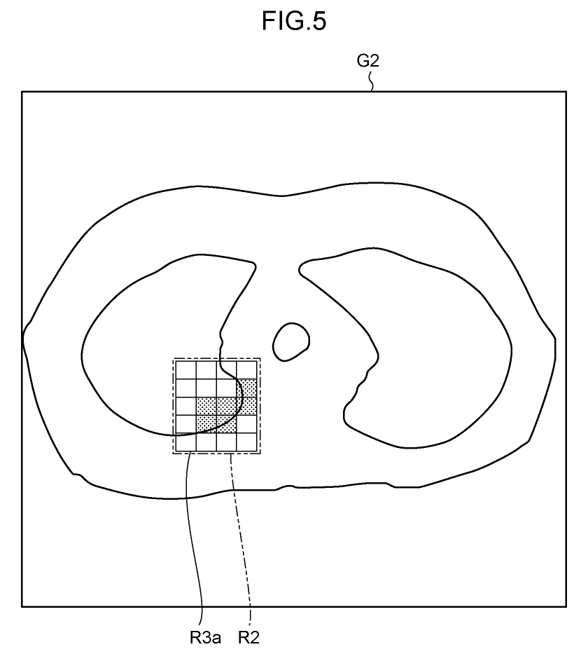

FIG. 5 is a diagram illustrating an example of the generated image G2 in which the generated region R2 can be identified. The output control function 353 may display a likelihood notification image R3a indicating the likelihood of each subregion of the generated region R2 by color. For example, when the likelihood is divided into multiple stages, the output control function 353 may display the likelihood notification image R3a indicating each subregion in a color corresponding to the stage to which the likelihood of the subregion belongs.

The output control function 353 may display the likelihood notification image R3a that expresses the likelihood by color shades. Furthermore, the output control function 353 may display the likelihood notification image R3a that expresses the likelihood not only by color but also by text, marks, or other methods.

### Second Embodiment

A medical information processing apparatus 30b in a medical information processing system 1b according to a second embodiment extracts the generated region R2, based on the difference between the input image data input to the generative model 21 and the generated image data output from the generative model 21.

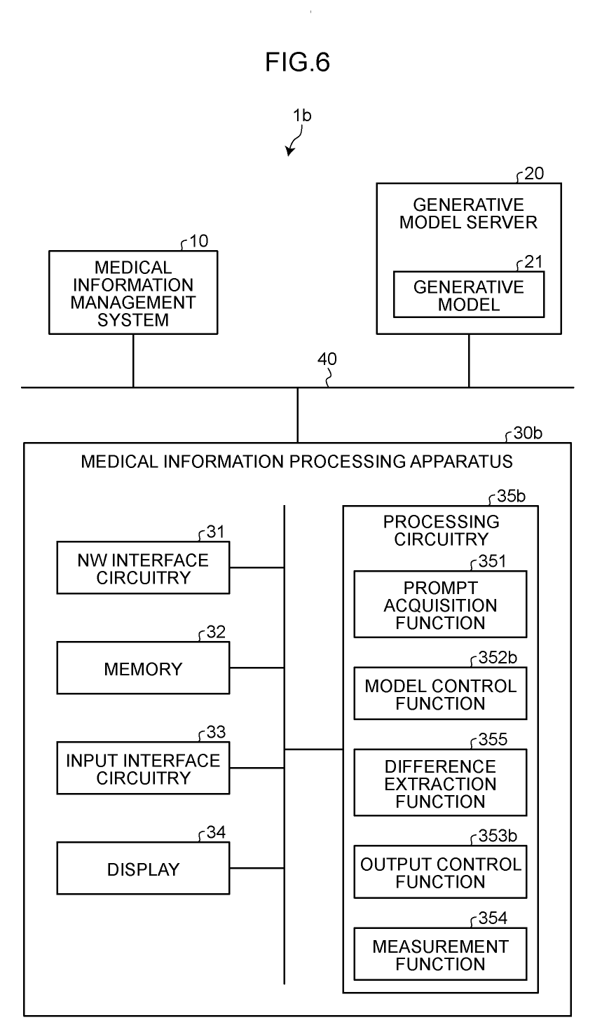

FIG. 6 is a diagram illustrating an example of the medical information processing system 1b according to the second embodiment. Processing circuitry 35b includes the prompt acquisition function 351, a model control function 352b, a difference extraction function 355, an output control function 353b, and the measurement function 354.

The prompt acquisition function 351 and the measurement function 354 have functions similar to those of the first embodiment.

The model control function 352b inputs the prompt acquired by the prompt acquisition function 351 into the generative model 21. The model control function 352b acquires the generated image data generated by the generative model 21 by inputting the prompt.

The difference extraction function 355 extracts the generated region R2 indicating the region where an image has been generated by the generative model 21, based on the difference between the input image data input to the generative model 21 and the generated image data output from the generative model 21. For example, the difference extraction function 355 executes alignment between the input image data stored in the memory and the generated image data stored in the memory, based on feature points such as anatomical landmarks. The difference extraction function 355 calculates the difference between the respective pixels at corresponding positions in the aligned images. The difference extraction function 355 then extracts a pixel with a difference equal to or greater than a threshold. Here, the difference extraction function 355 extracts the generated region R2 by considering the pixels with a difference equal to or greater than a threshold as being a region generated by the generative model 21. In other words, the difference extraction function 355 extracts the shape of the generated region R2 and the location of the generated region R2.

The output control function 353b displays the generated image G2 in which the generated region R2 can be identified on the display 34, based on the difference extracted by the difference extraction function 355. In other words, the output control function 353b displays the generated image G2 in which the generated region R2 can be identified, without using the image generation likelihood information.

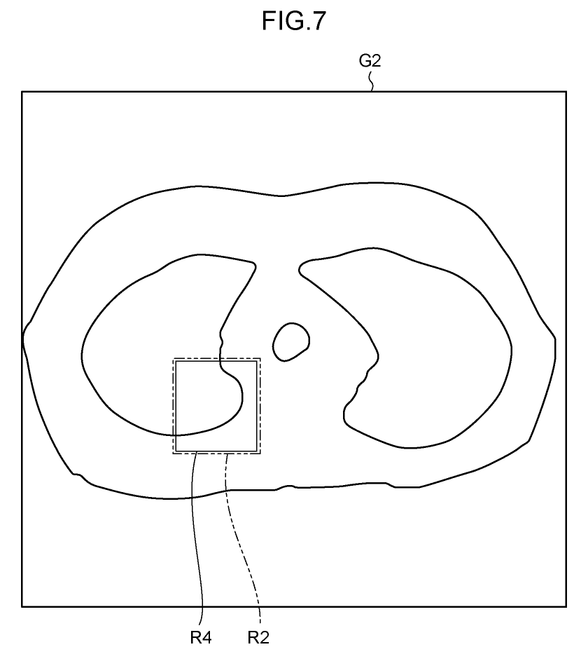

FIG. 7 is a diagram illustrating an example of the generated image G2 in which the generated region R2 can be identified. For example, the output control function 353b displays a generated notification image R4 of the same shape as the generated region R2 on the generated region R2, based on the difference extracted by the difference extraction function 355. The generated notification image R4 is an image that gives a notification of the shape of the generated region R2 and the location of the generated region R2. The generated notification image R4 does not give a notification of the likelihood of each subregion. However, the medical professional can grasp by the generated notification image R4 that the region is a region in which the image has been generated by the generative model 21.

The output control function 353b displays that the region is a region in which the image has been generated by the generative model 21, in an identifiable manner. Here, the generative model 21 generates an image of the image generation region R1. The output control function 353b then displays the generated region R2, which is the region of the image generated by the generative model 21, in an identifiable manner.

As described above, the medical information processing apparatus 30b according to the second embodiment warns when the measurement range is included in the generated region R2 generated by the generative model 21 in the generated image data. The medical professional, receiving the warning, can grasp that the image is an image generated by the generative model 21. Thus, when a medical professional uses an image, the medical information processing apparatus 30b can allow the medical professional to easily grasp that the image or a part of the image has been generated by the generative model 21.

The medical professional may cancel the measurement if a warning appears that the measurement range is included in the generated region R2 generated by the generative model 21 in the generated image data. In this case, the medical information processing apparatus 30b can reduce processor or memory usage.

Furthermore, as illustrated in FIG. 7, a medical information processing apparatus 30c displays the generated notification image R4 that gives a notification of the shape of the generated region R2 and the location of the generated region R2. The medical information processing apparatus 30 therefore can allow the medical professional to grasp the region generated by the generative model 21.

### Third Embodiment

The medical information processing apparatus 30c in a medical information processing system 1c according to a third embodiment warns when the measurement range is included in the generated region R2.

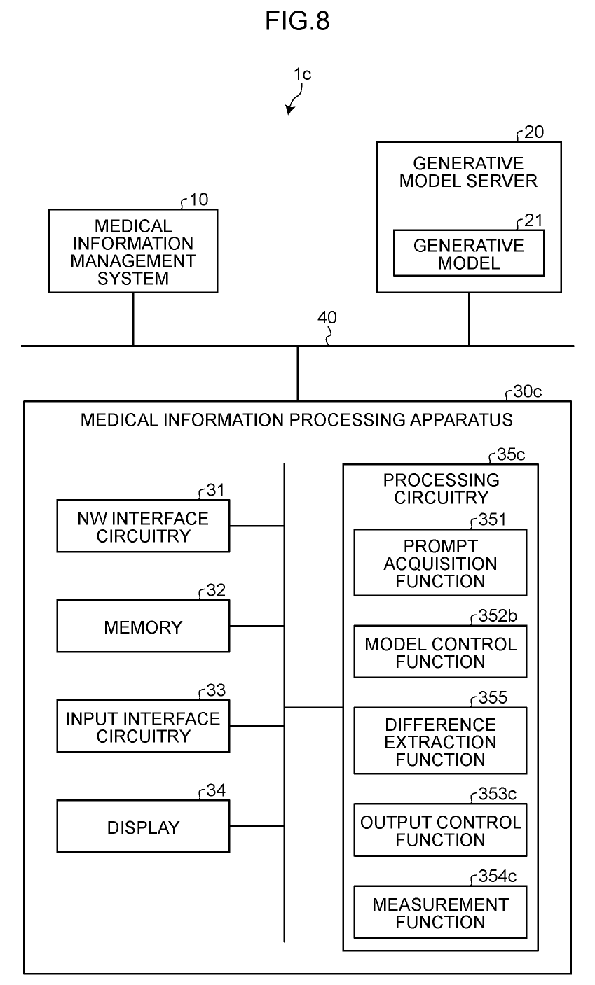

FIG. 8 is a diagram illustrating an example of the medical information processing system 1c according to the third embodiment. Processing circuitry 35c includes the prompt acquisition function 351, the model control function 352b, the difference extraction function 355, an output control function 353c, and a measurement function 354c.

The prompt acquisition function 351, the model control function 352b, and the difference extraction function 355 have functions similar to those of the second embodiment. The processing circuitry 35c illustrated in FIG. 8 may include the model control function 352 that has a function similar to that of the first embodiment. Furthermore, the processing circuitry 35c does not necessarily have the difference extraction function 355.

The output control function 353c displays the generated notification image R4 of the same shape as the generated region R2 on the generated region R2, based on the difference between the input image data extracted by the difference extraction function 355 and the generated image data.

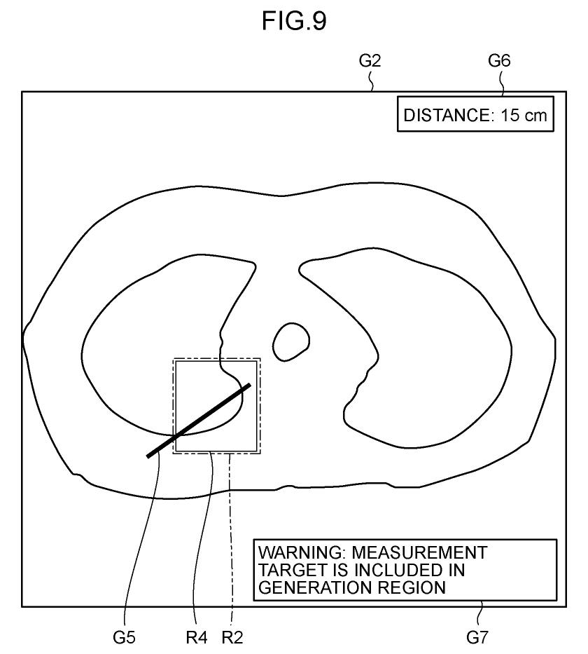

FIG. 9 is a diagram illustrating exemplary measurement using the generated image G2. The measurement function 354c accepts an operation to specify the measurement range in the generated image G2. For example, the measurement function 354c accepts an operation to specify the range including the generated region R2 as the measurement range. For example, the measurement function 354c measures the measurement range specified by a measurement tool called caliper G5 illustrated in FIG. 9. For example, the measurement function 354c measures the distance from one end to the other end of caliper G5.

When the measurement range is specified by the measurement function 354c, the output control function 353c displays a measurement result image G6 indicating the specified measurement range. For example, the measurement result image G6 is an image indicating the distance from one end to the other end of caliper G5.

Here, caliper G5 illustrated in FIG. 9 measures the measurement range including the generated region R2 highlighted by the generated notification image R4. The output control function 353c displays a warning image G7 if the measurement range of the measurement function 354c is included in the generated region R2. The warning image G7 is an image that warns that the measurement range of the measurement function 354c is included in the generated region R2. A medical professional can notice that the generated region R2 is being measured because the warning image G7 appears.

As illustrated in FIG. 9, the output control function 353c displays the generated notification image R4 that makes the generated region R2 identifiable. However, the output control function 353c may display the likelihood notification images R3 and R3a instead of the generated notification image R4. In this case, the output control function 353c still displays the warning image G7 if the measurement range of the measurement function 354c is included in the generated region R2.

As described above, the medical information processing apparatus 30c according to the third embodiment displays the warning image G7 when the measurement range is included in the generated region R2 generated by the generative model 21 in the generated image data. The medical professional, receiving the warning, can grasp that the image is an image generated by the generative model 21. Thus, when a medical professional uses an image, the medical information processing apparatus 30c can allow the medical professional to easily grasp that the image is an image generated by the generative model 21.

### First Modification

The output control functions 353, 353b, and 353c display the instruction content information included in the prompt on the display 34.

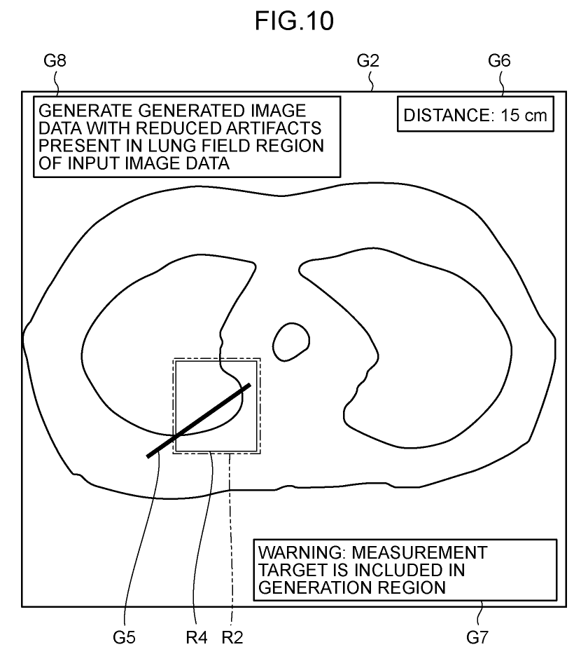

FIG. 10 is a diagram illustrating exemplary measurement using the generated image G2. As illustrated in FIG. 10, the output control functions 353, 353b, and 353c display a prompt image G8 indicating a prompt that has been used to instruct the generative model 21 to generate generated image data. For example, the output control functions 353, 353b, and 353c display the prompt image G8 at a position that does not overlap the generated region R2. Thus, the output control functions 353, 353b, and 353c do not prevent a medical professional from viewing the prompt image G8.

The prompt image G8 is an image indicating the instruction content information included in the prompt to give an instruction to generate generated image data. In other words, the output control functions 353, 353b, and 353c display the instruction contents for the generative model 21 as indicated by the instruction content information acquired by the prompt acquisition function 351. For example, the prompt image G8 is an image with text indicating the instruction contents for the generative model 21, such as "Generate generated image data with reduced artifacts present in the lung field region of the input image data".

Here, the prompt image G8 contains the phrase "artifacts present in the lung field region". In other words, the prompt image G8 has artifact location information indicating the location where artifacts are present. The prompt image G8 allows the medical professional to grasp the location where the generated region R2 is present. The prompt image G8 does not necessarily include artifact location information, such as "Generate generated image data with reduced artifacts present in the input image data". Furthermore, the prompt image G8 may give a notification of the location where the generated region R2 is present not only by the subject's part but also by other expression methods such as coordinates.

The output control functions 353, 353b, and 353c may change the display form of the warning image G7 based on the instruction content information included in the prompt. For example, if the artifact location information included in the instruction content information differs from the location where the generated region R2 is present, the generative model 21 may have made a mistake in setting the image generation region R1. Therefore, the output control functions 353, 353b, and 353c highlight the displayed warning image G7 if the artifact location information included in the instruction content information differs from the location where the generated region R2 is present. For example, the output control functions 353, 353b, and 353c change the color of the displayed warning image G7. The output control functions 353, 353b, and 353c may highlight the displayed warning image G7 not only by changing the color of the warning image G7 but also by displaying the warning image G7 with blinking or with an added icon or by other methods.

As illustrated in FIG. 10, the output control functions 353, 353b, and 353c display the generated notification image R4 that makes the generated region R2 identifiable. However, the output control functions 353, 353b, and 353c may display the likelihood notification images R3 and R3a instead of the generated notification image R4. In this case, the output control functions 353, 353b, and 353c still display the warning image G7 if at least a part of the measurement range of the measurement functions 354 and 354c is included in the generated region R2.

### Second Modification

The output control functions 353, 353b, and 353c display that the measurement range is normal if the measurement range is not included in the generated region R2.

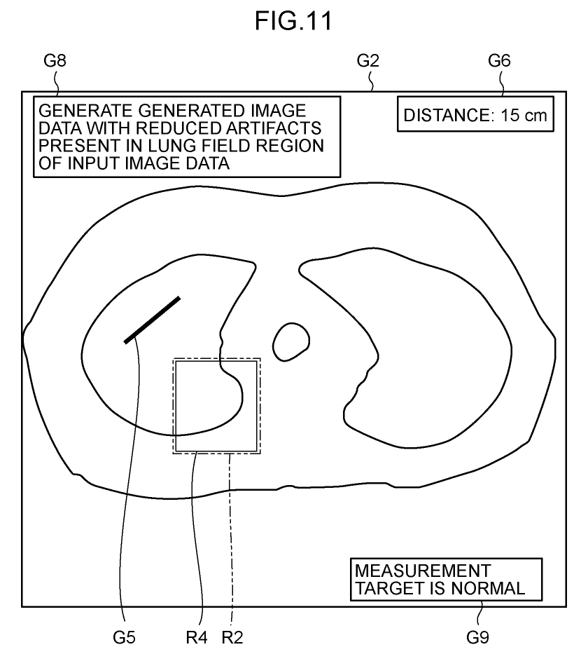

FIG. 11 is a diagram illustrating exemplary measurement using the generated image G2. As illustrated in FIG. 11, the output control functions 353, 353b, and 353c display a normal image G9 indicating that the measurement range is normal if the measurement range of the measurement functions 354 and 354c is not included in the generated region R2. In other words, the normal image G9 is an image that gives a notification that the image generated by the generative model 21 does not include the measurement range. For example, the output control functions 353, 353b, and 353c display the normal image G9 when the measurement range using caliper G5 is not included.

As illustrated in FIG. 11, the output control functions 353, 353b, and 353c display the generated notification image R4 that makes the generated region R2 identifiable. However, the output control functions 353, 353b, and 353c may display the likelihood notification images R3 and R3a instead of the generated notification image R4. In this case, the output control functions 353, 353b, and 353c still display the normal image G9 if the measurement range of the measurement functions 354 and 354c is not included in the generated region R2.

### Third Modification

The measurement functions 354 and 354c limit the measurement of the measurement range if at least a part of the measurement range is included in the generated region R2.

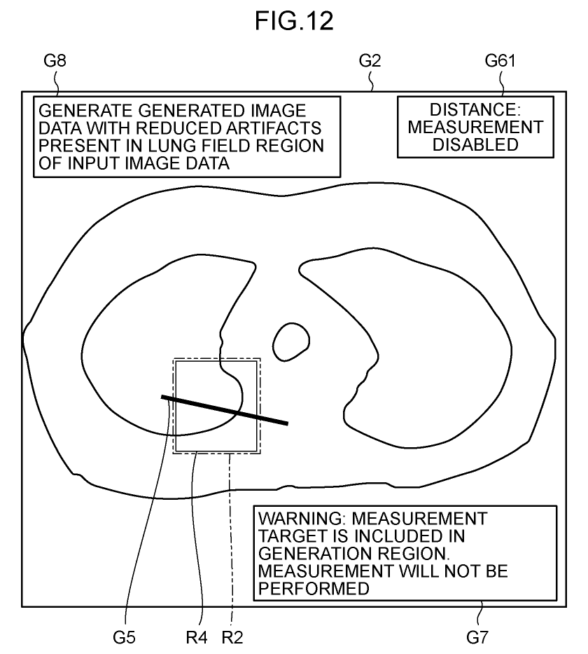

FIG. 12 is a diagram illustrating exemplary measurement using the generated image G2. The measurement functions 354 and 354c limit the measurement of the measurement range if at least a part of the measurement range is included in the generated region R2. As an example of limiting the measurement, the measurement functions 354 and 354c do not measure the measurement range.

Limiting the measurement is not limited to not measuring the measurement range. For example, the measurement functions 354 and 354c may limit the measurement according to accuracy. For example, the measurement functions 354 and 354c may perform measurements in centimeters but do not necessarily perform measurements in millimeters. The measurement functions 354 and 354c may limit the measurement depending on the purpose and type of inspection.

The output control functions 353, 353b, and 353c display a measurement-disabled image G61 indicating that the measurement is not allowed when it is determined that the measurement range is not to be measured by the measurement functions 354 and 354c. The measurement-disabled image G61 is an image that gives a notification that the measurement is not allowed because at least a part of the measurement range is included in the generated region R2.

### Fourth Modification

The measurement functions 354 and 354c limit the measurement of the measurement range if the likelihood of a subregion of the generated region R2 including the measurement range is less than a threshold.

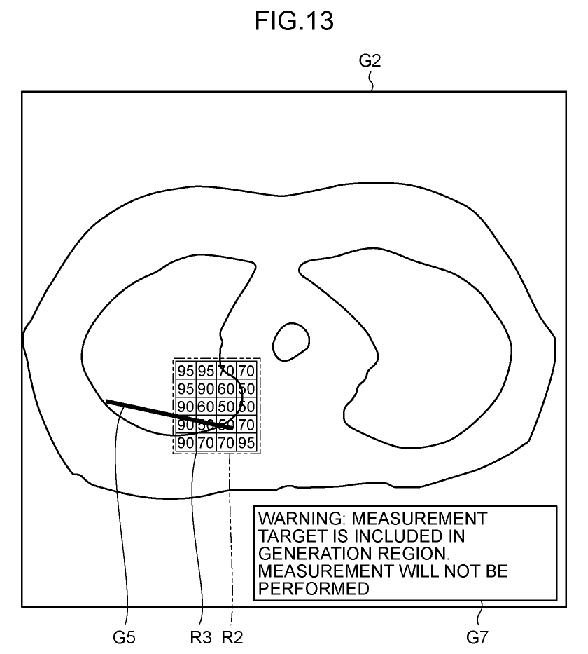

FIG. 13 is a diagram illustrating exemplary measurement using the generated image G2. The model control function 352 acquires the image generation likelihood information having the likelihood of an image generated by the generative model 21 for each of a plurality of subregions included in the generated region R2. The model control function 352 is an example of a likelihood acquisition unit. The measurement functions 354 and 354c limit the measurement of the measurement range if the likelihood of a subregion of the generated region R2 including the measurement range is less than a threshold. In other words, the measurement functions 354 and 354c limit the measurement of the measurement range if the likelihood of a subregion of the image generated by the generative model 21 is less than a threshold. As an example of limiting the measurement, the measurement functions 354 and 354c do not measure the measurement range.

Limiting the measurement is not limited to not measuring the measurement range. For example, the measurement functions 354 and 354c may limit the measurement according to accuracy. For example, the measurement functions 354 and 354c may perform measurements in centimeters but do not necessarily perform measurements in millimeters. The measurement functions 354 and 354c may limit the measurement depending on the purpose and type of inspection.

On the other hand, the measurement functions 354 and 354c measure the measurement range if the likelihood of a subregion including the measurement range in the generated region R2 is equal to or greater than the threshold. In other words, the measurement functions 354 and 354c measure the measurement range if the likelihood of a subregion of the image generated by the generative model 21 is equal to or greater than the threshold.

The output control functions 353, 353b, and 353c display the measurement-disabled image G61 indicating that the measurement is not allowed when it is determined that the measurement range is not to be measured by the measurement functions 354 and 354c. The measurement-disabled image G61 is an image that gives a notification that the measurement is not to be performed because the measurement range includes a region with low likelihood.

On the other hand, the output control functions 353, 353b, and 353c display the normal image G9 when it is determined that the measurement range is to be measured by the measurement functions 354 and 354c.

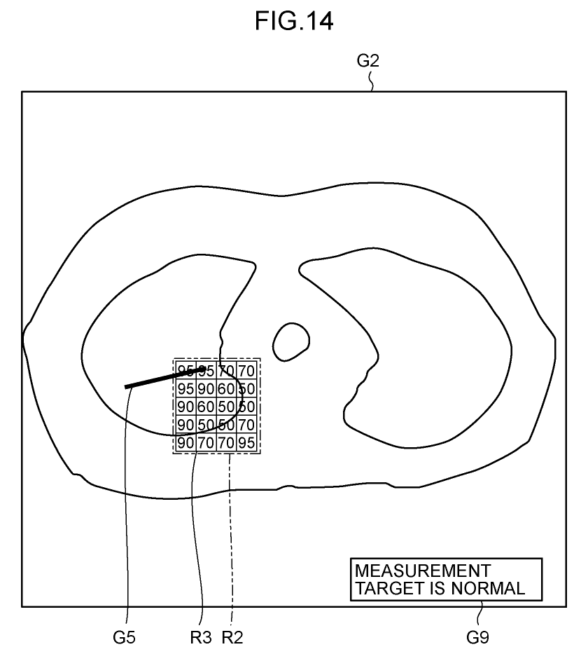

FIG. 14 is a diagram illustrating exemplary measurement using the generated image G2. As illustrated in FIG. 14, the output control functions 353, 353b, and 353c display the normal image G9 to give a notification that the measurement range is normal. The output control functions 353, 353b, and 353c may display the normal image G9 to give a notification that the measurement range is normal because the likelihood of the subregion of the generated region R2 including the measurement range is equal to or greater than a threshold.

### Fifth Modification

The output control functions 353, 353b, and 353c display a warning not only for two-dimensional medical image data, but also for three-dimensional medical image data.

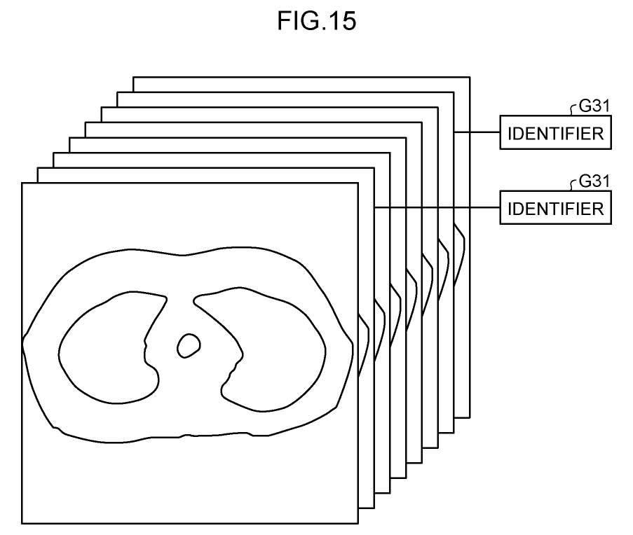

FIG. 15 is a diagram illustrating exemplary measurement using three-dimensional medical image data. The model control function 352 acquires three-dimensional medical image data in which at least one slice image is the generated image data. In other words, the model control function 352 acquires three-dimensional medical image data including the generated image data generated by the generative model 21. The model control function 352 may acquire the image generation likelihood information if the image generation likelihood information is output from the generative model 21.

The measurement function 354 accepts an operation to specify a measurement range. In this case, the measurement function 354 may accept the specification of a measurement range that includes a plurality of slice images. In other words, the measurement functions 354 and 354c measure a three-dimensional measurement range included in three-dimensional generated region notification image data.

The output control functions 353, 353b, and 353c add an identifier G31 to displayed two-dimensional generated image data that includes a subregion with likelihood lower than a threshold when the three-dimensional measurement range by the measurement functions 354 and 354c is included in the subregion with likelihood lower than the threshold. A medical professional thus can grasp whether the measurement range is included in the image generated by the generative model 21, even in the three-dimensional generated region notification image data.

### Sixth Modification

In the description above, the output control functions 353, 353b, 353c output predetermined information when the measurement range measured by the measurement functions 354 and 354c is included in the generated region R2 of the generated image data generated by the generative model 21. However, the output control functions 353, 353b, and 353c may output predetermined information not only when the measurement range is included in the generated region R2 but also when the generated region R2 is used in other ways. For example, the output control functions 353, 353b, and 353c may output predetermined information when an arrow or the like directed to the generated region R2 is added, or when a color is added to highlight a region including the generated region R2, or when captured in a reading report.

### Seventh Modification

In the description above, the model control functions 352 and 352b input input image data to the generative model 21 and acquire the generated image data generated by the generative model 21. The data to be input to the generative model 21 is not limited to CT image data generated by reconstruction processing, but may be data before reconstruction processing. The data before reconstruction processing may be raw data detected by an X-ray detector or may be projection data that has undergone preprocessing such as logarithmic transformation, offset correction, sensitivity correction between channels, and beam hardening correction. In this case, the output control functions 353, 353b, and 353c still input the data before reconstruction processing to the generative model 21 and output predetermined information if at least a part of the measurement range is included in the generated region R2 in the generated image data generated by the generative model 21.

### Eighth Modification

The medical information processing system 1 according to the first embodiment has been described as having the medical information management system 10, the generative model server 20, and the medical information processing apparatus 30. The medical information processing apparatus 30 may be an X-ray CT apparatus.

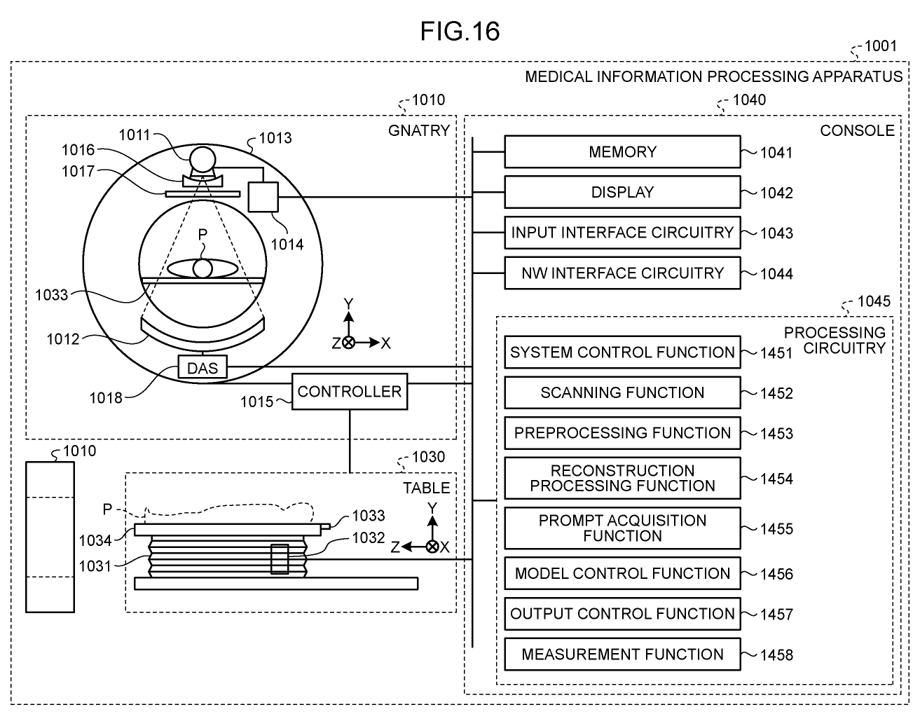

FIG. 16 is a diagram illustrating an exemplary configuration of a medical information processing apparatus 1001 according to an eighth modification. The medical information processing apparatus 1001, which is an X-ray CT apparatus, has a gantry 1010, a table 1030, and a console 1040.

In FIG. 16, the rotational axis of a rotation frame 1013 in a non-tilted state or the longitudinal direction of a table-top 1033 of the table 1030 is defined as the Z-axis direction. The axis direction orthogonal to the Z-axis direction and horizontal to the floor surface is defined as the X-axis direction. The axis direction orthogonal to the Z-axis direction and the X-axis direction and perpendicular to the floor surface is defined as the Y-axis direction. FIG. 16 illustrates the gantry 1010 depicted from multiple directions for illustration and illustrates a case where the medical information processing apparatus 1001 has one gantry 1010.

The gantry 1010 has an X-ray tube 1011, an X-ray detector 1012, the rotation frame 1013, X-ray high voltage circuitry 1014, a controller 1015, a wedge 1016, a collimator 1017, and a data acquisition system (DAS) 1018. The gantry 1010 is also referred to as a gantry.

The X-ray tube 1011 is a vacuum tube having a cathode (filament) that generates thermal electrons and an anode (target) that generates X-rays upon collision of the thermal electrons. The X-ray tube 1011 emits thermal electrons from the cathode toward the anode with a high voltage applied from the X-ray high voltage circuitry 1014 to irradiate the subject P with X-rays. In other words, the X-ray tube 1011 generates X-rays in accordance with a tube voltage and a tube current applied from the X-ray high voltage circuitry 1014. For example, the X-ray tube 1011 includes a rotating anode-type X-ray tube that generates X-rays by applying thermal electrons to a rotating anode.

The X-ray detector 1012 detects X-rays emitted from the X-ray tube 1011 and passing through the subject P, and outputs a signal corresponding to the detected X-ray dose to the DAS 1018. The X-ray detector 1012 has, for example, a plurality of detector element rows having a plurality of detector elements arranged in the channel direction (channel direction) along one arc around the focus of the X-ray tube 1011. The X-ray detector 1012 has, for example, a structure in which a plurality of detector element rows having a plurality of detector elements arranged in the channel direction are arranged in the row direction (slice direction, row direction).

For example, the X-ray detector 1012 is an indirect conversion type detector having a grid, a scintillator array, and an optical sensor array. The scintillator array has a plurality of scintillators. The scintillators have a scintillator crystal that outputs light with a photon quantity corresponding to an incident X-ray dose. The grid has an X-ray shielding plate disposed on a surface of the scintillator array on the X-ray incidence side to absorb scattered X-rays. The grid may also be called collimator (one-dimensional collimator or two-dimensional collimator). The optical sensor array has a function of converting light into an electrical signal corresponding to the light quantity from the scintillator and, for example, has an optical sensor such as a photodiode. The X-ray detector 1012 may be a direct conversion type detector having a semiconductor device that converts incident X-rays into electrical signals.

The rotation frame 1013 is an annular frame that supports the X-ray tube 1011 and the X-ray detector 1012 such that they face each other, and rotates the X-ray tube 1011 and the X-ray detector 1012 by the controller 1015. For example, the rotation frame 1013 is a casting made of aluminum. In addition to the X-ray tube 1011 and the X-ray detector 1012, the rotation frame 1013 can further support the X-ray high voltage circuitry 1014, the wedge 1016, the collimator 1017, the DAS 1018, and the like. The rotation frame 1013 can further support various components not illustrated in FIG. 16. The various components supported by the rotation frame 1013 will be described below. The rotation frame 1013 is also referred to as rotating base or rotating body.

The X-ray high voltage circuitry 1014 has a high voltage generator that has electrical circuitry such as a transformer and a rectifier to generate a high voltage to be applied to the X-ray tube 1011, and an X-ray controller that controls an output voltage in accordance with the X-rays generated by the X-ray tube 1011. In other words, the X-ray high voltage circuitry 1014 controls a tube voltage and a tube current applied to the X-ray tube 1011. The high voltage generator may be a transformer system or an inverter system. The X-ray high voltage circuitry 1014 may be provided in the rotation frame 1013 or in a not-illustrated stationary frame.

The controller 1015 has processing circuitry having a central processing unit (CPU) or the like and a drive mechanism such as a motor and an actuator. The controller 1015 receives an input signal from input interface circuitry 1043 and performs operation control for the gantry 1010 and the table 1030. For example, the controller 1015 controls the rotation of the rotation frame 1013, the tilt of the gantry 1010, and the movement of the table 1030 and the table-top 1033. The controller 1015 may be provided in the gantry 1010 or in the console 1040.

The wedge 1016 is a filter for regulating the dose of X-rays emitted from the X-ray tube 1011. Specifically, the wedge 1016 is a filter that transmits and attenuates the X-rays emitted from the X-ray tube 1011 so that the X-rays emitted from the X-ray tube 1011 toward the subject P have a predetermined distribution. For example, the wedge 1016 is a filter, such as a wedge filter or a bow-tie filter, formed by processing aluminum or the like to have a predetermined target angle and a predetermined thickness.

The collimator 1017 is a lead plate or the like to narrow the irradiation range of X-rays transmitted through the wedge 1016 and forms a slit by combining a plurality of lead plates or the like. The collimator 1017 may also be called an X-ray aperture. Although FIG. 16 illustrates a case where the wedge 1016 is disposed between the X-ray tube 1011 and the collimator 1017, the collimator 1017 may be disposed between the X-ray tube 1011 and the wedge 1016. In this case, the wedge 1016 transmits and attenuates X-rays emitted from the X-ray tube 1011 with the irradiation range limited by the collimator 1017.

The DAS 1018 collects a signal of X-ray detected by each detector element of the X-ray detector 1012. For example, the DAS 1018 has an amplifier that performs amplification processing on an electrical signal output from each detection element and an A/D converter that converts the electrical signal into a digital signal to generate detection data.

The data generated by the DAS 1018 is transmitted from a transmitter having a light emitting diode (LED) in the rotation frame 1013 to a receiver having a photodiode in a non-rotating part (e.g., stationary frame not illustrated in FIG. 16) of the gantry 1010 via optical communication, and then transferred to the console 1040. Here, the non-rotating part is, for example, a stationary frame that supports the rotation frame 1013 in a rotatable manner. The method of transmitting the data from the rotation frame 1013 to the non-rotating part of the gantry 1010 is not limited to optical communication, and any non-contact type data transfer method or contact type data transfer method may be employed.

The table 1030 is an apparatus on which the subject P to be imaged is placed and transferred, and has a base 1031, a couch driver 1032, the table-top 1033, and a support frame 1034. The base 1031 is a housing that supports the support frame 1034 such that the support frame 1034 can be moved vertically. The couch driver 1032 is a drive mechanism that moves the table-top 1033 on which the subject P is placed in the long-axis direction of the table-top 1033, and includes a motor and an actuator. The table-top 1033 provided on the upper surface of the support frame 1034 is a plate on which the subject P is placed. In addition to the table-top 1033, the couch driver 1032 may move the support frame 1034 in the long-axis direction of the table-top 1033.

The console 1040 has a memory 1041, a display 1042, the input interface circuitry 1043, network (NW) interface circuitry 1044, and processing circuitry 1045. The console 1040 is described as a separate unit from the gantry 1010, but the gantry 1010 may include the console 1040 or a part of each component of the console 1040.

The memory 1041 is implemented, for example, by a semiconductor memory device such as random access memory (RAM) or flash memory, a hard disk, or an optical disc. The memory 1041 stores therein, for example, projection data and CT image data. For example, the memory 1041 stores therein a computer program for circuitry included in the medical information processing apparatus 1001 to implement various functions. The memory 1041 may be implemented by a group of servers (cloud) connected to the medical information processing apparatus 1001 via the network 40.

The display 1042 displays various information. For example, the display 1042 displays various images generated by the processing circuitry 1045 or displays a graphical user interface (GUI) for accepting various operations from the operator. For example, the display 1042 is a liquid crystal display or a cathode ray tube (CRT) display. The display 1042 may be a desktop type or may be configured as a tablet terminal or the like capable of wireless communication with a main body of the console 1040.

The input interface circuitry 1043 is implemented by a mouse, a keyboard, a trackball, a switch, a button, a joystick, a touchpad that allows for an input operation by touching an operation surface, a touchscreen that integrates a display screen and a touchpad, a non-contact input circuit using an optical sensor, an audio input circuit, and the like. The input interface circuitry 1043 may be provided on the gantry 1010. The input interface circuitry 1043 may be configured as a tablet terminal or the like capable of wireless communication with the main body of the console 1040. The input interface circuitry 1043 is not limited to those with physical operating components, such as a mouse and a keyboard. For example, an electrical signal processing circuit that receives an electrical signal corresponding to an input operation from an external input device installed separately from the console 1040 and outputs the electrical signal to the processing circuitry 1045 is also an example of the input interface circuitry 1043.

The NW interface circuitry 1044 is connected to the processing circuitry 1045 and controls transmission and communication of various data to and from each apparatus connected via the network 40. For example, the NW interface circuitry 1044 is implemented by a network card, a network adapter, a network interface controller (NIC), or the like.

The processing circuitry 1045 controls the operation of the entire medical information processing apparatus 1001. For example, the processing circuitry 1045 executes a system control function 1451, a scanning function 1452, a preprocessing function 1453, a reconstruction processing function 1454, a prompt acquisition function 1455, a model control function 1456, an output control function 1457, and a measurement function 1458. For example, each of the processing functions executed by the system control function 1451, the scanning function 1452, the preprocessing function 1453, the reconstruction processing function 1454, the prompt acquisition function 1455, the model control function 1456, the output control function 1457, and the measurement function 1458, which are the components of the processing circuitry 1045 illustrated in FIG. 16, is stored in the memory 1041 in the form of a computer program executable by a computer. The processing circuitry 1045 is, for example, a processor that reads computer programs from the memory 1041 and executes the computer programs to implement a function corresponding to each of the computer programs. In other words, the processing circuitry 1045 in a state in which the computer programs are read out has each function depicted in the processing circuitry 1045 in FIG. 16.

In FIG. 16, a single processor implements the processing functions performed by the system control function 1451, the scanning function 1452, the preprocessing function 1453, the reconstruction processing function 1454, the prompt acquisition function 1455, the model control function 1456, the output control function 1457, and the measurement function 1458. However, a plurality of independent processors may be combined to construct the processing circuitry 1045 so that each processor executes a computer program to implement a function. In FIG. 16, the single memory 1041 stores therein the computer program corresponding to each processing function. However, a plurality of memories 1041 may be distributed and allocated, and the processing circuitry 1045 may be configured to read a corresponding computer program from each individual memory 1041.

The system control function 1451 controls various functions of the processing circuitry 1045 based on an input operation received from the operator via the input interface circuitry 1043. The system control function 1451 is an example of a control unit.

The scanning function 1452 controls the gantry 1010 according to the acquired inspection order information. With this configuration, the scanning function 1452 scans the subject P.

The preprocessing function 1453 generates data by performing preprocessing such as logarithmic transformation, offset correction, sensitivity correction between channels, and beam hardening correction on the detection data output from the DAS 1018. The data before preprocessing (detection data) and the data after preprocessing may be collectively referred to as projection data. The preprocessing function 1453 is an example of a preprocessing unit.

The reconstruction processing function 1454 generates CT image data by performing reconstruction processing on the projection data generated by the preprocessing function 1453, using filtered back projection, iterative reconstruction, or the like. In other words, the reconstruction processing function 1454 generates CT image data based on the detection data detected by the X-ray detector 1012. The reconstruction processing function 1454 is an example of a reconstruction processing unit. The reconstruction processing function 1454 converts the CT image data generated by the reconstruction processing function 1454 into any cross-sectional tomographic image data or any three-dimensional image data by a known method, based on an input operation accepted from the operator via the input interface circuitry 1043. The three-dimensional image data may be generated directly by the reconstruction processing function 1454.

The prompt acquisition function 1455 has a function similar to the prompt acquisition function 351, except that the CT image data generated by the reconstruction processing function 1454 is used as input image data. In more detail, the prompt acquisition function 1455 controls the input interface circuitry 1043 to acquire a prompt indicating instruction contents for the generative model 21.

The prompt includes instruction content information and instruction target information. The instruction target information is information indicating the CT image data generated by the reconstruction processing function 1454 as the input image data serving as an instruction target. For example, the instruction target information is identification information to identify the CT image data, which is the input image data serving as an instruction target, or a path indicating the location where the CT image data, which is the input image data, is saved. The instruction target information may be the CT image data itself, which is the input image data. In this case, the prompt acquisition function 1455 acquires a prompt including medical image data.

The model control function 1456 has a function similar to the model control function 352. In more detail, the model control function 1456 inputs the prompt acquired by the prompt acquisition function 1455 into the generative model 21. The model control function 1456 acquires the generated image data output from the generative model 21. The model control function 1456 is an example of an image acquisition unit. For example, the model control function 1456 acquires the generated image data in which artifacts contained in the input image data are reduced by the generative model 21 that generates an image with reduced artifacts.

The output control function 1457 has a function similar to the output control function 353. In more detail, the output control function 1457 outputs predetermined information when at least a part of the measurement range of the measurement function 1458 is included in the generated region R2 in the generated image data. For example, the output control function 1457 displays the generated image G2 on the display 34 based on the generated image data. For example, the output control function 1457 displays the generated image G2 in which the generated region R2, which is the region of the image generated by the generative model 21, can identified. For example, the output control function 353 displays the likelihood notification image R3 of the same shape as the generated region R2 on the generated region R2, based on the image generation likelihood information.

The measurement function 1458 has a function similar to the measurement function 354. In more detail, the measurement function 1458 measures the measurement range using the generated image G2, based on an operation accepted by the input interface circuitry 1043. The measurement function 1458 is an example of a measurement unit. In more detail, the measurement function 1458 accepts the specification of the measurement range in the generated image G2, based on an operation accepted by the input interface circuitry 1043. The measurement function 1458 then measures the measurement range included in the generated image data.

As described above, the medical information processing apparatus 1001 according to the fourth embodiment is an X-ray CT apparatus. In this case, like the medical information processing apparatus 30 according to the first embodiment, when a medical professional uses an image, the medical information processing apparatus 1001 allows the medical professional to easily grasp that the image or a part of the image has been generated by the generative model 21. Furthermore, the medical information processing apparatus 1001 can also display the likelihood of each subregion by means of the likelihood notification image R3. The medical information processing apparatus 1001 therefore can allow the medical professional to grasp the likelihood of the image generated by the generative model 21.

The processing circuitry 1045 has been described as having functions similar to those of the processing circuitry 1045 of the medical information processing apparatus 30 according to the first embodiment. The processing circuitry 1045 may have functions similar to those of the modifications and other embodiments.

The output control function 1457 may have a function similar to that of the first modification of the first embodiment.

The prompt acquisition function 1455 may have a function similar to the prompt acquisition function 351 according to the second embodiment. The model control function 1456 may have a function similar to the model control function 352b according to the second embodiment. The output control function 1457 may have a function similar to the output control function 353b according to the second embodiment. The measurement function 1458 may have a function similar to the measurement function 354 according to the second embodiment. The processing circuitry 1045 may have a function similar to the difference extraction function 355 according to the second embodiment.

The prompt acquisition function 1455 may have a function similar to the prompt acquisition function 351 according to the third embodiment. The model control function 1456 may have a function similar to the model control function 352b according to the third embodiment. The output control function 1457 may have a function similar to the output control function 353c according to the third embodiment. The measurement function 1458 may have a function similar to the measurement function 354c according to the third embodiment. The processing circuitry 1045 may have a function similar to the difference extraction function 355 according to the third embodiment.

The output control function 1457 may have a function similar to the output control functions 353, 353b, and 353c according to the first modification of the third embodiment.

The output control function 1457 may have a function similar to the output control functions 353, 353b, and 353c according to the second modification of the third embodiment.

The measurement function 1458 may have a function similar to the measurement functions 354 and 354c according to the third modification of the third embodiment.

The measurement function 1458 may have a function similar to the measurement functions 354 and 354c according to the fourth modification of the third embodiment.

The output control function 1457 may have a function similar to the output control functions 353, 353b, and 353c according to the fifth modification of the third embodiment.

The output control function 1457 may have a function similar to the output control functions 353, 353b, and 353c according to the sixth modification of the third embodiment.

The output control function 1457 may have a function similar to the output control functions 353, 353b, and 353c according to the seventh modification of the third embodiment.

According to at least one of the embodiments and the like described above, when a medical professional performs a measurement using an image, the medical professional can easily grasp that the image or a part of the image has been generated by the generative model 21.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray CT apparatus comprising:
an X-ray tube configured to generate an X-ray;
an X-ray detector configured to detect the X-ray emitted from the X-ray tube and passing through a subject; and
processing circuitry, wherein
the processing circuitry is configured to
generate medical image data based on detection data detected by the X-ray detector,
input the medical image data into a generative model and acquire generated image data generated by the generative model,
measure a measurement range using the generated image data, and
in a case where at least a part of the measurement range is included in a generated region in the generated image data, output predetermined information.

2. A medical image processing apparatus comprising processing circuitry configured to
input medical image data into a generative model and acquire generated image data generated by the generative model,
measure a measurement range using the generated image data, and
in a case where at least a part of the measurement range is included in a generated region in the generated image data, output predetermined information.

3. The medical image processing apparatus according to claim 2, wherein the generated region of the generated image data includes image data generated by the generative model.

4. The medical image processing apparatus according to claim 2, wherein in the case where at least a part of the measurement range is included in the generated region in the generated image data, the processing circuitry outputs information regarding re-measurement to a measurement unit.

5. The medical image processing apparatus according to claim 2, wherein the generated region of the generated image data is at least one of: a region where artifacts are reduced in the medical image data; a region where information outside an imaging region is generated in the medical image data; and a region where a device related to treatment is removed in the medical image data.

6. The medical image processing apparatus according to claim 2, wherein the processing circuitry displays likelihood of an image generated by the generative model for each of a plurality of subregions included in the generated region.

7. The medical image processing apparatus according to claim 6, wherein the processing circuitry displays a prompt that has been used to instruct the generative model to generate the generated image data.

8. The medical image processing apparatus according to claim 2, wherein the processing circuitry displays an image indicating that the measurement range is a normal measurement range in a case where the measurement range is not included in the generated region.

9. The medical image processing apparatus according to claim 2, wherein the processing circuitry does not measure the measurement range in the case where the measurement range is included in the generated region.

10. The medical image processing apparatus according to claim 2, wherein
the processing circuitry
acquires likelihood of an image generated by the generative model for each of a plurality of subregions included in the generated region, and
in a case where the measurement range is included in the subregion with the likelihood less than a threshold, does not measure the measurement range.

11. The medical image processing apparatus according to claim 10, wherein the processing circuitry measures the measurement range in a case where the likelihood of the subregion that includes the measurement range in the generated region is equal to or greater than a threshold.

12. A medical image processing method comprising:
inputting medical image data into a generative model and acquiring generated image data output from the generative model;
measuring a measurement range using the generated image data; and
in a case where at least a part of the measurement range is included in a generated region in the generated image data, outputting predetermined information.
